(19) United States
(12) Patent Application Publication
(10) Pub. No.: US 2020/0330208 A1
(43) Pub. Date: Oct. 22, 2020

(54) THERAPEUTIC COVERING FOR HORSES

(71) Applicant: NAAR BOVEN CORPORATION, Ocala, FL (US)

(72) Inventor: Katherine L. Chrysostom, Ocala, FL (US)

(73) Assignee: NAAR BOVEN CORPORATION, Ocala, FL (US)

(21) Appl. No.: 16/897,948

(22) Filed: Jun. 10, 2020

Related U.S. Application Data
(63) Continuation of application No. 15/221,244, filed on Jul. 27, 2016, now abandoned.
(60) Provisional application No. 62/211,305, filed on Aug. 28, 2015.

(51) Int. Cl. A61D 9/00; A01K 13/00; A61N 2/06; A61F 7/00

(52) U.S. Cl. CPC A61D 9/00; A61F 7/0097; A61N 2/06; A01K 13/008

(57) ABSTRACT

A therapeutic covering comprises a blanket comprising mesh infused with ceramic mineral powder (emitting far-infrared wavelengths) comprising approximately 80-150 magnets over the primary acupuncture points. These magnets are neodymium magnets with a strength ranging from approximately 1100-2400 gauss. The blanket is sold in a range of sizes to fit ponies and horses alike. The magnets themselves are sewn into neoprene pockets and cannot be moved from their original sewn-in position.

### CROSS-REFERENCE TO RELATED APPLICATIONS

This application is a continuation of U.S. Non-provisional Patent Application Ser. No. 15/221,244, filed on Jul. 27, 2016, which claims the benefit of U.S. Provisional Patent Application Ser. No. 62/211,305, filed Aug. 28, 2015, the contents of which applications are herein incorporated by reference in their entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file, but otherwise reserves all copyright rights whatsoever. 37 FR 1.71(d).

### FIELD OF THE INVENTION

The present invention relates generally to the field of magnetic and ceramic textile therapies and more specifically relates to a therapeutic scrim for equine use comprising ceramic textile and, in addition, has static neodymium magnets located on top of specific acupuncture points of the horse to provide a combination therapy for stimulation or healing of soft tissue injuries and wounds, reduction of inflammation and soreness, suppleness of muscles, increased blood flow, relaxation and prophylactic measures.

### BACKGROUND OF THE INVENTION

The following includes information that may be useful in understanding the present invention(s). It is not an admission that any of the information provided herein is prior art, or material, to the presently described or claimed inventions, or that any publications or document that is specifically referenced is prior art.

A scrim is a type of covering, generally speaking, a large piece of mesh, intended to keep the user's temperature regulated, regardless of moisture level. Scrims can be distinguished from sheets by their thickness and purpose; the thickest scrim is still thinner than the lightest sheet. Scrims are generally used for drying an equine after bathing or for help keeping flies and other insects away from the animal, while sheets are for hygiene, comfort and aesthetics.

A neodymium magnet is a rare-earth magnet made from an alloy of neodymium, iron and boron to form the NdzFe14B tetragonal crystalline structure. Neodymium magnets are the strongest type of permanent magnet commercially available.

Magnet therapy, magnetic therapy, or magnotherapy has been accepted as a safe alternative medicine practice by involving the use of static or pulsing magnetic fields to derive beneficial health effects such as oxygenating soft tissues, stimulating blood flow in underlying tissues, improve overall immunity, and creating the harmonization of bodily functions. Magnet therapy is the application of the magnetic field of electromagnetic devices or permanent static magnets to the body for health benefits.

The material used to compose the textile potion of this therapeutic covering is infused with Ceramic powder which is a combination of thirty (30) Lead-free minerals including but not limited to Silica [SiO2], Magnesium [Mg], and Aluminum [Al]. The Ceramic textile can often be referred to as “far-infrared clothing”. Ceramic textile emits far-infrared wavelengths which stimulates vibration of the Oxygen atom within the water molecules (H2O). As the Oxygen vibrates around the Hydrogen atoms, the water molecules begin to shrink in size which then stimulates the increase in blood circulation as well as dilation of blood vessels. This process also lends a helping hand to the body in development of new capillaries (branching blood vessels with a hair-like thickness) for treatment of deep muscle lesions or soft tissue injuries.

The combination of ceramic textile and static magnetic therapy offers a unique, safe, and non-invasive therapy. The therapeutic benefits of this combination therapy can be used as pre-exercise and/or post-exercise functions. This scrim helps to “warm-up” muscles and relax tension before exercise or as a post-exercise therapy, this scrim will help to eliminate soreness, reduce inflammation, and increase blood circulation for enhanced healing and recovery.

Various attempts have been made to solve problems found in blankets solely utilizing magnets. Among these are found in: U.S. Pat. No. 5,984,855 to Lisanne DiNapoli; U.S. Pat. No. 5,426,925 to Larita A. Smargiassi; and U.S. Pat. No. 5,888,185 to Michael T. Regan. This prior art is representative of blankets comprising magnets.

None of the above inventions and patents, taken either singly or in combination, is seen to describe the invention as claimed. Thus, a need exists for a reliable therapeutic covering, a scrim created for equine use comprising ceramic material and static neodymium magnets targeting acupuncture points on the horse to provide therapy and to avoid the above-mentioned problems.

### SUMMARY OF THE INVENTION

In view of the foregoing disadvantages inherent in the known scrim comprising at least one magnet device art, the present invention provides a novel therapeutic covering. The general purpose of the present invention, which will be described subsequently in greater detail, is to provide a scrim comprising ceramic material and static neodymium magnets located on acupuncture points of the horse to provide a safe and non-invasive therapy. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, a therapeutic covering, constructed and operative according to the teachings of the present invention.

FIG. 1 is a side view illustrating a therapeutic covering according to an embodiment of the present invention.

FIG. 2 is a perspective view illustrating a therapeutic covering according to an embodiment of the present invention in FIG. 1.

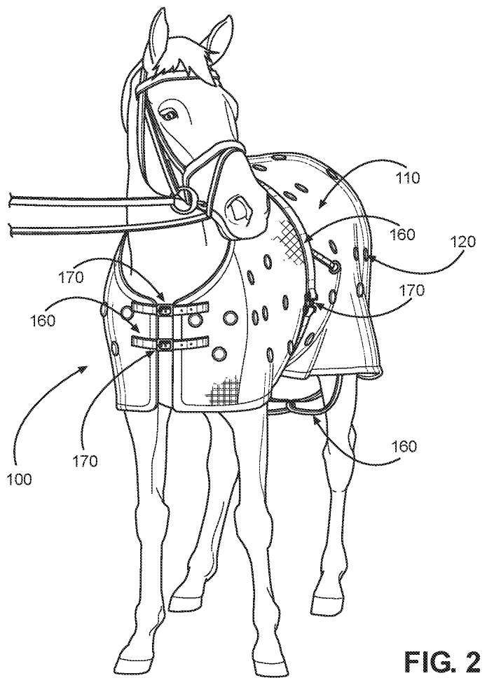

FIG. 3 is a view illustrating a pocket of the therapeutic covering having a magnet therein according to an embodiment of the present invention in FIG. 1.

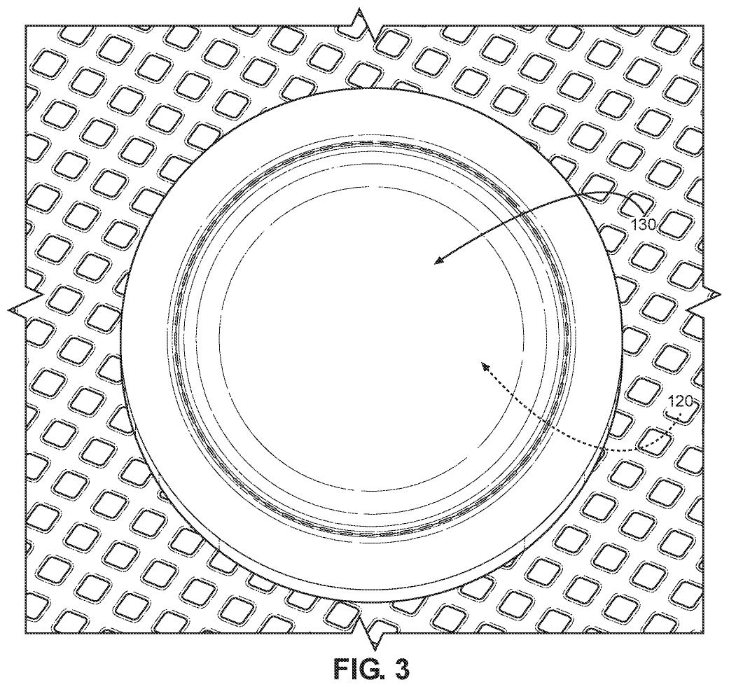

The various embodiments of the present invention will hereinafter be described in conjunction with the appended drawings.

### DETAILED DESCRIPTION OF PREFERRED EMBODIMENTS

As discussed above, embodiments of the present invention relate to a blanket comprising at least one magnet device and more particularly to a therapeutic covering, a scrim created for equine use comprising ceramic textile and static neodymium magnets located on acupuncture points of the horse to provide a non-invasive therapy.

The therapeutic covering comprises a novel product offering consumers a practical solution to the aforementioned challenges. As the name implies, the therapeutic covering comprises a specially designed covering consisting of mesh material infused with Ceramic mineral powder (emitting far-infrared wavelengths) comprising approximately 80-150 magnets over the primary acupuncture points. These magnets are neodymium magnets with a strength ranging from approximately 1100-2400 gauss. The scrim is sold in a range of sizes to fit ponies and horses alike. The magnets themselves are sewn into neoprene pockets and cannot be moved from their original sewn-in position.

The magnets are placed over the primary acupuncture points including, but not limited to: release points that line the vertebrae, ulcer-prone areas and stomach, shoulders, haunches and rib cages. The magnets are specifically placed to further enhance what the ceramic textile therapy is accomplishing. The therapeutic covering is cost-effective to produce in the embodiments.

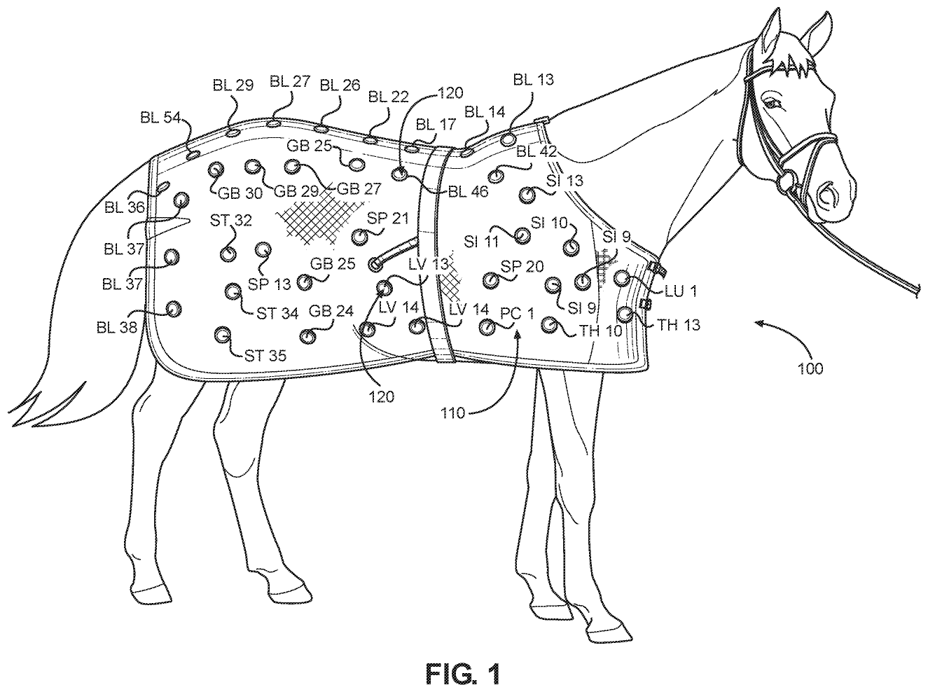

Referring now to the drawings FIGS. 1 -3, the therapeutic covering is formed as a therapeutic covering 100 for horses comprising a mesh material 110 adapted to cover a portion of a horse's body and is infused with ceramic mineral powder, and a plurality of magnets 120 attached to said mesh material at predetermined locations that are adapted to correspond to predetermined acupuncture points of horses. The mesh material 110 further comprises a plurality of pockets 130 attached thereto at the predetermined locations, and wherein each of the plurality of pockets are adapted to retain a respective one of the plurality of magnets 120 therein. The plurality of pockets 130 may be lined with neoprene material, and the plurality of magnets 120 may be formed from a neodymium material. The pluralities of magnets 120 have a strength range of between 1100 to 2400 gauss, and in the preferred embodiment the number of magnets used is between 80 to 150. When produced for use, the plurality of pockets 130 comprises an equal number to the number of magnets.

The ceramic mineral powder may be formed as a combination of lead-free materials including, but not being limited to, silica, magnesium, and aluminum.

The plurality of attachment members 160 are connected to the mesh material and are adapted to connect spaced portions of the mesh material such that the mesh material can be secured in place upon the horse's body. The plurality of attachment members can be formed as adjustable strap members having releasable connectors 170 attached thereto.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A therapeutic covering for a horse comprising:
a fabric material adapted to cover a portion of a body of said horse, said fabric material being infused with ceramic mineral powder; and
a plurality of magnets attached to said fabric material at predetermined locations corresponding to predetermined acupuncture points of said horse.

2. The therapeutic covering of claim 1, wherein said fabric material further comprises a plurality of pockets attached thereto at said predetermined locations; and wherein each of said plurality of pockets retains a respective one of said plurality of magnets therein.

3. The therapeutic covering of claim 2, wherein said plurality of pockets are lined with neoprene material.

4. The therapeutic covering of claim 1, wherein said plurality of magnets are formed from a neodymium material.

5. The therapeutic covering of claim 1, wherein said plurality of magnets have a strength range of between 1100 to 2400 gauss.

6. The therapeutic covering of claim 1, wherein said plurality of magnets comprise between 80 to 150 magnets.

7. The therapeutic covering of claim 2, wherein said plurality of magnets comprise a first number between 80 to 150 magnets and said plurality of pockets comprise a second number equal to said first number of said plurality of magnets.

8. The therapeutic covering of claim 1, wherein said ceramic mineral powder comprises a combination of lead-free materials chosen from a list of lead-free materials consisting of silica, magnesium, and aluminum.

9. The therapeutic covering of claim 1, further comprising a plurality of attachment members connected to spaced portions of said fabric material and connectable to each other across said body of said horse with said fabric material placed thereon.

10. The therapeutic covering of claim 9, wherein said plurality of attachment members are formed as adjustable strap members including releasable connectors attached thereto.

11. The therapeutic covering of claim 1, wherein said fabric material is a mesh material.
